(19) United States
(12) Patent Application Publication
(10) Pub. No.: US 2006/0034872 A1
(43) Pub. Date: Feb. 16, 2006

(54) Compositions and methods for preventing abuse of orally administered medications

(76) Inventor: Clifford J Woolf, Newton, MA (US)

Correspondence Address: CLARK & ELBING LLP, 101 FEDERAL STREET, BOSTON, MA, 02110, US

(21) Appl. No.: 10/510,266

(22) PCT Filed: Apr. 23, 2003
(86) PCT No.: PCT/US03/12496

§ 371 Date: Apr. 21, 2005

Related U.S. Application Data
(60) Provisional application No. 60/376,147, filed on Apr. 29, 2002.

(51) Int. Cl. A61K 36/81; A61K 31/5513; A61K 31/47; A61K 9/00; A61K 31/202; A61K 31/165

(52) U.S. Cl. 424/400; 424/760; 514/617; 514/310; 514/221; 514/560

(57) ABSTRACT

Disclosed herein is the use of chemical irritants, such as vanilloid receptor-1 agonists, in sustained/controlled release pharmaceutical preparations which also contain a drug typically having high abuse potential. Inclusion of the VR1 agonist in the pharmaceutical preparation interferes with illicit or inappropriate dosing without significantly interfering with the action of the therapeutic. Also disclosed are exemplary co-formulations of capsaicin (a VR1 agonist) and oxycodone (an opioid therapeutic having high abuse potential) in controlled release preparations.

### FIELD OF THE INVENTION

In general, the invention relates to new formulations that reduce the addiction or abuse potential of opioids and other orally administered therapeutics.

### BACKGROUND OF THE INVENTION

Opioids are among the most potent known analgesics and, when used correctly, are generally safe. However, opioids do possess very strong reinforcing properties and, if administered improperly, can be abused. Repetitive abuse or use can result in addiction (physical and psychological dependence). Typically, abuse arises from self-administration in the absence or in excess of a medical need. Opioid addiction, for example, arises either from repetitive abuse or repetitive use for therapeutic purposes.

The abuse or addictive properties of opioids stem from their rapid penetration into the nervous system and stimulation of opiate receptors. Activation of these receptors in the mesolimbic brain structures, including the ventral tegmental area, nucleus accumbens, and hippocampus, appears to result in excessive dopamine release in the nucleus accumbens causing intense euphoria. In addition to the forebrain, opiate receptors are also present in the brainstem, spinal cord and gastrointestinal tract. Addiction results both from the attempt to maintain euphoria as well as the avoidance of withdrawal effects that result from terminating excessive opioid use. Tolerance develops from the repetitive use of opioids which reduces the euphoric effects and induces dosage escalation by the abuser.

Adverse effects and even death can result from opioid overdose. Mild or moderate opioid intoxication can result in intestinal symptoms such as decreased gastric motility (constipation) and nausea. Sedation and pruritis (itching) are also common adverse effects. More severe opioid overdoses can inhibit respiration by activating the opiate receptors of the brain stem, resulting in respiratory failure. Thus, there is a need to develop formulations which permit administration of opioids in safe and therapeutic dosages while, at the same time, limiting the abuse potential of this powerful class of narcotics.

### SUMMARY OF THE INVENTION

The present invention provides new drug formulations and methods for reducing addiction and abuse potential. In particular, the invention features a pharmaceutical composition including (i) a therapeutic compound and (ii) an irritant molecule such as a vanilloid receptor-1 (VR1) agonist. Also featured is a method for controlling administration of a therapeutic compound by mixing the therapeutic compound with a VR1 agonist in the same pharmaceutical composition. Further, a method for manufacturing a pharmaceutical composition that includes a therapeutic compound and a VR1 agonist is provided. Generally, these methods and compositions control the frequency, route, and dose of therapeutic self-administration and deter illicit administration.

In preferred embodiments of the compositions and methods of the invention, the pharmaceutical composition is formulated for controlled release (CR) and is suitable for ingestion. Further, the pharmaceutical composition is prepared such that its destruction or tampering renders the VR1 agonist available for immediate release following administration by any route.

In particularly useful compositions and methods, the therapeutic compound is one which has a high abuse potential such as an opioid, a barbiturate, a cannabinoid, an amphetamine, or a benzodiazepine. Opioids that are particularly suited for combination with a VR1 agonist including, for example, morphine, oxycodone, hydrocodone, hydromorphone, levorphanol, buprenorphine, butorphanol, fentanyl, dipipanone, codeine, dihydrocodeine, tramadol, etorphine, dihydroetorphine, meperidine, methadone, propoxyphene, and heroin. Most preferably, the opioid is oxycodone, oxymorphone, or morphine.

Any VR1 agonist is useful in the method and compositions of this invention including, for example, resiniferanoids, capsaicinoids, phorboid vanilloids, and terpenoid 1,4-unsaturated dialdehydes. Specifically, useful compounds include, for example, capsaicin, resiniferatoxin, olvanil, piperine, zingerone, anandamide, 12- and 15-(S)-hydroperoxy-eicosatetraenoic acids, 5- and 15-(S)-hydroxyeicosatetraenoic acids, phorbol 12-phenylacetate 13-acetate 20-homovanillate, 2 phorbol 12,13-didecanoate 20-homovanillate, leukotriene B(4), N-(3-acyloxy-2-benzylpropyl)-N′-dihydroxytetrahydrobenzazepine, and tetrahydroisoquinoline thiourea analogs. Particularly useful VR1 agonists include capsaicin, resiniferatoxin, and olvanil.

The therapeutic compound used in the present invention can be present in amounts known to be clinically effective. The VR1 agonist used in the compositions and methods of this invention are in doses equivalent to at least about the maximum tolerated dose, or about two times, three times, four times, five times, or even six times the maximum tolerated dose for an average person (70 kg). Therefore, at least about 1-2000 μg, preferably 10-1500 μg, and more preferably 25-1000 μg of capsaicin may be present in a controlled release formulation. About 0.1-500 μg, preferably 0.5-250 μg, and more preferably 1-100 μg of resiniferatoxin may be present in each controlled release formulation. About 1-5000 μg, preferably 5-2500 μg, and more preferably 10-1000 μg of olvanil may be present in each controlled release formulation. Equipotent doses of other VR1 agonists can also be used.

By “therapeutic compound” is meant any medicament administered for the purpose of treating (i.e., preventing, reducing, or eliminating) a medical condition. Such medical conditions can include pain disorders as well as physical, emotional, and psychiatric diseases or disorders, may be temporary or permanent, and may have any etiology that is known or unknown.

By “therapeutic having high abuse potential” is meant any medically useful therapeutic which, when administered improperly, results in physical and/or psychological dependence (i.e., addiction). Typically, the addictive properties result from, and are reinforced by, a euphoric sensation caused by a supra-therapeutic dose or a rapid rise of plasma levels. Frequently abused therapeutics include, for example, opioids (i.e., oxycodone, morphine, hydrocodone, methadone, codeine, and meperidine), barbiturates, cannabinoids, amphetamines, and benzodiazepines.

By “vanilloid receptor-1 agonist” or “VR1 agonist” is meant any compound which is capable of binding to a vanilloid receptor-1 (VR1), for example, a human VR1, and eliciting a biological effect attributable to such binding. VR1 binding is easily measured using receptor binding techniques known in the art of receptor pharmacology. Assays include, for example, ligand displacement assays against known VR1 ligands. Preferably, VR1 agonists bind with high affinity, having a dissociation constant (K_{d})<10 μM, <1 μM, <500 nM, <100 nM, <10 nM, <1 nM, or even <100 pM. Biological effects of VR1 agonists can be measured either in vitro or in vivo using techniques which are well known in the art (see, for example, Szallasi and Blumberg, Pharmacol. Rev., 51:159-211, 1999). Several specific techniques for measuring the biological effect of VR1 agonists are provided below.

By “pharmaceutical composition” is meant a composition containing a therapeutic compound which is suitable for administration to a subject (i.e., patient). The most useful pharmaceutical compositions, for the purposes of the invention, are those which are suitable for ingestion (oral administration). Such compositions can be formulated for controlled/sustained release or immediate release of the therapeutic. Pharmaceutical compositions can be prepared by any method known in the art such as those described, for example, in Remington's Pharmaceutical Sciences, (19th edition), ed. A. Gennaro, 1995, Mack Publishing Company, Easton, Pa.

By “controlling administration” is meant a method for reducing or preventing the excessive, illicit, or improper administration of a therapeutic or pharmaceutical composition.

By “illicit administration” or “improper administration” is meant any administration of a pharmaceutical, intended for clinical use, which is not being administered according to instructions from a heath care professional or administered for medical need. Illicit administration of medically useful therapeutics may be a result of addiction and the administered dose is typically greater than clinically or therapeutically indicated. Alternatively, improper or illicit administration is done by changing the route of therapeutic administration. Typically, orally administered therapeutics are taken by a buccal, intranasal, or intravenous route generating a rapid rise in plasma levels and inducing euphoric or psychotomimetic reinforcing effect. By “formulated for controlled release” is meant the formulation of any pharmaceutical preparation for prolonged or sustained duration of release and delivery of a compound (i.e., a therapeutic). Typically, a controlled release formulation contains two, three, four, or more times the total amount of the compound than is normally present in an “immediate release” formulation and is administered as an alternative to a course of multiple dose therapy. Controlled release may minimize the reinforcing effects of therapeutic compounds having high abuse potential by reducing the rate of rise of plasma concentration, thereby preventing the euphoria-inducing effects.

By “irritant molecule” is meant any substance which has the potential to cause pain, discomfort, or non-life threatening physiological effect following administration, by any route, to a mammal (e.g., a human) by activating high threshold sensory fibers (nociceptors). Irritant molecules can be naturally occurring or synthetic and can produce the irritating effect either directly, for example, by interacting with a receptor expressed on a nociceptor, or indirectly.

### DETAILED DESCRIPTION

Many opioids, including oxycodone, have very short biological and therapeutic half-lives. The introduction of controlled/sustained release (CR) opioid formulations has enabled development of simpler pain management and dosing regimens. Presently, CR formulations of oxycodone, morphine, hydromorphone, and hydrocodone are available but are frequently abused because of the high amounts of opioid contained in each dosage unit. The present invention provides methods and compositions which deter illicit administration of opioids resulting from tampering with CR formulations. Specifically, an irritant molecule, such as a VR1 agonist like capsaicin, is included in the opioid-containing CR formulation. Ingesting the undamaged CR formulation results in the gradual release of the VR1 agonist which can be metabolized and excreted without discomfort to the patient. Destruction of the CR formulation and administration by any route results in transient but intense discomfort to the user sufficient to deter further inappropriate administration. This technique of including a VR1 agonist in a CR formulation can be used in conjunction with any therapeutic, not just opioids, and will deter illicit administration. Further, any VR1 agonist can be substituted for capsaicin in the compositions and methods of the invention.

Controlled Release Formulations

The disadvantage of widespread use of CR formulations is that each dosage unit contains many times the opioid content included in immediate release (IR) formulations. For example, current formulations of OxyContin® (oxycodone) are designed for 12 hour slow release and contains 2-32 times as much oxycodone as the IR formulations. Thus, any tampering with the CR delivery vehicle (e.g., pill) can liberate a euphoria-inducing, potentially addictive, and potentially lethal opioid dose. Currently, simple destruction of the CR pill by crushing allows the potential abuser to access large amounts of the opioid which can be administered orally, intranasally, or intravenously. The high bioavailability of oxycodone, in particular, by oral and intranasal administration makes it attractive for abusers wanting to avoid hypodermic injections. Currently there is no effective means for preventing tampering or premature release of increased opioid doses contained within a CR formulation. Opioid manufacturers and the FDA are including “Black Box” warnings on the package insert which warn of serious events leading to significant injury or death, in an attempt to prevent inappropriate prescribing and administration. While these warnings may deter legitimate medical users from excessive medication, they do little to prevent illicit administration.

Controlled release opioid formulations which incorporate an opioid receptor antagonist have been proposed as a means of limiting the tampering and abuse of the CR formulations. However, this strategy is frequently limited by differences in the pharmacokinetic profile of the receptor antagonist and the drug of abuse. For example, the opioid antagonist naloxone can be co-formulated with oxycodone. Naloxone, however, is not absorbed following oral or intranasal administration. Therefore, while preventing opioid-induced euphoria following intravenous injection, naloxone fails to block the addictive effects of oxycodone when administered by other more common routes such as ingestion or “snorting.” Furthermore, using antagonists targeted to the same receptor as the therapeutic reduces the desired clinical action of the therapeutic compound, (i.e., the analgesic opioid effect).

Although it is possible that orally available opioid receptor antagonists may be developed, the antagonist will only be applicable to opioid-containing CR formulations. Thus, the technique of co-formulating a low affinity ligand having the opposite pharmacologic effect (i.e., an antagonist) in a CR preparation is not a viable solution to the general problem in cases where the therapeutic is not an opioid agonist. For example, by following this strategy, each class of addictive therapeutics would require its own antagonist. The development of novel antagonists would command significant drug discovery, safety, and efficacy testing resources. Moreover, many useful therapeutics interact with more than one molecular target or receptor and, frequently, the therapeutic benefit and/or addictive properties are mediated by several of these targets. Thus, a CR formulation may require the inclusion of several antagonists to block the reinforcing and addictive properties of the main therapeutic. In addition, it may be impossible to design antagonists with appropriate receptor affinities and pharmacokinetic properties which would effectively deter pill tampering. Further, because antagonists often have properties and chemical structures that are similar to agonists, co-administration may result in an undesirable interaction through, for example, competition for absorption, metabolism, or excretion. Thus, CR formulations are needed which do not interfere with therapy but deter medication tampering. The most preferable formulations are those which could be used in conjunction with any therapeutic that carries the potential for abuse.

Improved Control Release Formulations

The present invention provides CR pharmaceutical formulations that discourage tampering through the incorporation of a chemical irritant into the formulation. Preferred chemical irritants act only on receptors expressed on the sensory neurons of the skin, oral cavity, nasal cavity, throat, and rectum, but not those present in the lower esophagus, stomach, small or large intestine. Further, the most preferred chemical irritants, other than stimulating sensory nociceptors, have no other significant pharmacological effect. Thus, the CR formulation of the invention, when swallowed intact, would not cause any discomfort to the patient or interfere with the pharmacological action of the therapeutic. The irritant would be released with the therapeutic agent in the stomach or small intestine and be metabolized either in the gut or by first pass metabolism in the liver. Destruction by digestion or first pass metabolism must be sufficient such that neither the residual unmetabolized irritant nor its metabolites present in the feces cause rectal skin irritation.

Like the main therapeutically active pharmaceutical in the formulation, the irritant molecule should be released in a sustained manner, allowing adequate dilution and/or metabolism to occur without causing irritation. Crushing or otherwise tampering with the CR formulation, in addition to immediately releasing the therapeutic (i.e., opioid), also immediately releases the chemical irritant. It is essential, however, that the irritant does not itself cause major life-threatening effects beyond severe discomfort, pain, and, possibly, transient physiological effects, and does not interfere with the pharmacological effects of the therapeutic.

Vanilloid Receptor Agonists

Preferred improved CR formulations of the present invention make use of vanilloid receptor agonists. The VR1 subtype of the vanilloid receptor is localized on nociceptive afferent neurons in cutaneous nerves, urinary bladder, urethra, trachea and main bronchi, vagal nerve, and nasal musoca (reviewed in Szallasi et al., Pharmacol. Rev. 51:159-211, 1999). The tissue localization and mediation of noxious stimuli makes VR1 an ideal target for chemical irritants useful for preventing the tampering with and destruction of CR formulations.

The VR1 agonist, capsaicin, the pungent ingredient in red peppers of the genus Capsicum, and resiniferatoxin, an irritant diterpene present in the latex of several members of the genus Euphorbia, have all of the qualities required of chemical irritants for use as an abuse deterrent in CR formulations. The application of capsaicin or resiniferatoxin to the skin, oral or nasal mucosa, or subdermal tissue elicits a sensation of burning pain mediated by selective stimulation of the VR1 receptors of nociceptive afferent neurons. A burning pain sensation also follows the application of a dermal cream containing 0.075% capsaicin. Further, tongue and throat irritation occurs after ingestion of as little as 3 ppm capsaicin, and nasal irritation and rhinorrhea occurs with instillation of 75-150 μg.

Inhaled capsaicin (10⁻³-10⁻⁸M solutions) immediately results in sneezing, coughing, and airway constriction. Further stimulation of the bronchial VR1 triggers the pulmonary chemoreflex (Bezold-Jarisch Reflex) characterized by bradycardia, depressed respiration (dyspnoea or apnea), and hypotension. This reflex shows little or no desensitization upon repeated capsaicin challenge. These minor physiologic effects subside almost immediately upon cessation of capsaicin inhalation and are not life-threatening.

Capsaicin produces very severe pain when injected intradermally, but does not cause pain when injected directly into a vein (up to 650 μM). The paravenous tissue is, however, highly sensitive to low capsaicin doses (0.3-6.5 μM). Although intravenous capsaicin injection does not cause pain, it can trigger the pulmonary chemoreflex by activating pulmonary VR1 receptors. Further, capsaicin injection directly into the superior vena cava (>0.5 μg/kg) produces a raw, burning sensation in the chest, face, rectum, and extremities, consistent with activation of peripheral VR1 receptors. In humans, the maximum tolerated dose (MTD) is approximately 4 μg/kg.

Although capsaicin is highly pungent and produces numerous uncomfortable physiological responses through its interaction with the VR1 receptor, capsaicin toxicity is relatively low. For example, repeated instillation of intravesical capsaicin (1-2 mM) over a period of five years did not result in pathological tissue changes (Dasguptaa et al., Eur. Urol. 33: 28-31, 1998). Likewise, long term intranasal capsaicin (0.15 mg per nostril, every 2-3 days; total capsaicin dose=1.05 mg) produced no histological abnormalities (Blom et al. Clin. Exp. Allergy 28:1351-1358, 1998).

Relatively high doses of capsaicin are used in many cultures as a culinary supplement without any obvious adverse effects. Following oral administration, it is readily absorbed from the gastrointestinal tract and almost completely metabolized in the liver. The hepatic metabolites released into the general circulation (hepatic vein) do not possess capsaicin-like biological activity. Thus, ingestion of capsaicin carries little risk of producing systemic effects (Donner et al., Naunyn Schmied. Arch. Pharmacol. 342:357-361, 1990). Additionally, olvanil, a capsaicin analog with similar pungent properties, is also subject to first pass metabolism (Sietsema et al., Life Sci. 43:1385-1391, 1988).

Capsaicin and its analogs are therefore safe deterrents for use in CR formulations in combination with drugs having a high abuse potential. Pill tampering, in addition to liberating the entire dose of the main therapeutic, also releases the entire capsaicin dose which is sufficient to produce pain when ingested or administered intranasally. “Snorting” will also cause sneezing and coughing. Intravenous injection of the capsaicin bolus will cause pain if the injection is not completely within a vein having a high flow rate. Further, even if the injection is completely intravenous, the capsaicin will elicit pain in the chest, extremities, and face as well as induce coughing. Although desensitization can occur, this requires multiple exposure and is rapidly reversed unless there is chronic continuous exposure for many weeks. Apart from its action on the VR1 receptor, capsaicin appears to have only minimal or non-specific side effects, and has not been reported to produce any pathology in humans other than degeneration/withdrawal of peripheral sensory fibers after prolonged continuous exposure. Moreover, at doses that are likely to be a useful deterrent, there is no evidence of dangerous cardiovascular complications after administration by any route.

Controlled Release Oral Dosage Forms

Capsaicin, or any other suitable pungent chemical irritant (i.e., any other VR1 agonist), can be incorporated into any of the controlled release dosage forms known in the art. The most useful controlled release forms for the compositions of this invention are diffusion systems and osmotic systems.

Diffusion systems are typically either reservoir devices or matrix devices. Reservoir devices consist of a drug-containing core surrounded by a semi-permeable membrane. The release rate of the drug contained in the core of a reservoir device depends on the dissolution rate of the drug, the diffusion rate across the membrane, or both. Thus, the artisan has fine control over the release rate of the two molecules by varying the amount of capsaicin, the membrane material, and core matrix material. Typically, reservoir devices are created by microencapsulation techniques using barrier membranes of, for example, gelatin, methyl-, ethyl-, or polyhydroxycellulose, or polyvinylacetate. Matrix diffusion devices, which depend primarily on dissolution rates, are also useful for the purposes of this invention; however, they provide less flexibility for modifying the release characteristics of the capsaicin and the therapeutic.

Osmotic controlled release systems are typically formulated as either an osmotic tablet or a two-compartment system. Osmotic tablets consist of a drug-containing hypo-osmotic core surrounded by a rigid semi-permeable membrane. The core absorbs water through the membrane, causing the drug to be expelled through a delivery orifice in the membrane. The two-compartment system relies on similar principles; however, the osmotically-active component is separated from the drug-containing compartment by a movable partition. As the osmotically-active compartment absorbs water and swells, the contents of the drug compartment are expelled from the device.

Any controlled release formulation can be prepared according to the principles of this invention. Other useful controlled release formulations are known to one skilled in the art and described, for example, in Remington's Pharmaceutical Sciences, (19th edition), ed. A. Gennaro, 1995, Mack Publishing Company, Easton, Pa.

Other VR1 Receptor Agonists

Although capsaicin is described most extensively, equipotent doses of any VR1 receptor agonist with a similar efficacy, metabolism, and toxicity profile can be substituted. Other suitable VR1 receptor agonists include, without limitation, resiniferatoxin, olvanil, piperine, zingerone, anandamide, phorbol 12-phenylacetate 13-acetate 20-homovanillate, 2-phorbol 12,13-didecanoate 20-homovanillate, 12- and 15-(S)-hydroperoxyeicosatetraenoic acids, 5- and 15-(S)-hydroxyeicosatetraenoic acids, leukotriene B(4), N-(3-acyloxy-2-benzylpropyl)-N′-dihydroxytetrahydro-benzazepine, and tetrahydroisoquinoline thiourea analogs.

Identification of VR1 Receptor Agonists

VR1 is a non-selective cation channel, permeable to both monovalent and divalent cations. Accordingly, candidate VR1 agonists can be screened by measuring the cation conductance in preparations of cells which either naturally express VR1, or have been induced to express VR1, for example, by inserting a transgene. Of the divalent cations, VR1 is particularly permeable to Ca²⁺. Thus, VR1 activation is easily monitored in cultured cells using fluorescent calcium-sensitive dyes (e.g., Fura-2) or calcium flux assays. Alternatively, biological effects can be measured electrophysiologically in isolated neurons (e.g., dorsal root ganglion neurons) as agonist-evoked currents (see, for example, Liu et al., Neuroscience 84:569-581, 1998), or physiologically by measuring the activation of nociceptors in a test subject. When measured electrophysiologically, the application of a VR1 agonist useful in the present invention causes at least 50% of the maximum calcium current induced by capsaicin. Desirably, the VR1 agonist causes at least 50%, 75%, 100%, 150%, or 200% of the maximum calcium current.

The effects of VR1 agonists may also be measured electrophysiologically using a skin/nerve preparation (see, for example, Seno et al., Neuroscience 55: 563-569, 1993). VR1 agonists suitable for use in the methods and compositions of this invention cause a significant increase in mechano-heat receptor firing compared to the control condition. Preferably, the VR1 agonist is at least 50%, 75%, 100%, 150%, or 200% as effective as capsaicin in the skin/nerve preparation assay.

Alternatively, the efficacy of a potential VR1 agonist can be assessed using whole animal assays. For example, VR1 agonists should, like capsaicin, induce a nociceptive response and/or hyperalgesia to mechanical and thermal stimuli when injected into the rodent plantar hindpaw (see, for example, Gilchrist et al., Pain 67: 179-188, 1996). Nociceptive responses are observed as repeated hindpaw flinching. Typically, at least about 100-200 flinches are observed per injection of a VR-1 agonist at a dosage that is equipotent to about 30 μg per 10 μl of capsaicin. By contrast, injection of 10 μl of saline usually results in less than five flinches. The duration of nociceptive response is usually about 5-10 minutes. Hyperalgesia induced by VR1 agonists is observed as a decrease in withdrawal latency and/or an increased withdrawal response. Here again, it is preferably that any candidate VR1 agonist suitable for use in the methods and compositions of this invention be at least 50%, 75%, 100%, 150%, or 200% as effective as capsaicin.

### Other Embodiments

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A pharmaceutical composition comprising (i) a therapeutic compound, and (ii) a vanilloid receptor-1 (VR1) agonist.

2. The pharmaceutical composition of claim 1, wherein said composition is suitable for ingestion.

3. The pharmaceutical composition of claim 1, wherein said composition is formulated for controlled release.

4. The pharmaceutical composition of any of claim 1 wherein said VR1 agonist is selected from the group consisting of capsaicin, resiniferatoxin, olvanil, piperine, zingerone, anandamide, 12- and 15-(S)-hydroperoxy-eicosatetraenoic acids, 5- and 15-(S)-hydroxyeicosatetraenoic acids, phorbol 12-phenylacetate 13-acetate 20-homovanillate, 2 phorbol 12,13-didecanoate 20-homovanillate, leukotriene B(4), N-(3-acyloxy-2-benzylpropyl)-N′-dihydroxytetrahydrobenzazepine, and tetrahydroisoquinoline thiourea analogs.

5. The pharmaceutical composition of claim 4, wherein said VR1 agonist is capsaicin.

6. The pharmaceutical composition of claim 4, wherein said VR1 agonist is resiniferatoxin.

7. The pharmaceutical composition of claim 1, wherein said therapeutic compound is an opioid.

8. The pharmaceutical composition of claim 7, wherein said opioid is morphine, oxycodone, or hydrocodone.

9. The pharmaceutical composition of claim 1, wherein said therapeutic compound is selected from the group consisting of morphine, oxycodone, hydrocodone, hydromorphone, levorphanol, buprenorphine, butorphanol, fentanyl, dipipanone, codeine, dihydrocodeine, tramadol, etorphine, dihydroetorphine, meperidine, methadone, propoxyphene, and heroin.

10. The pharmaceutical composition of claim 1, wherein said therapeutic compound is a cannabinoid, an amphetamine, or a benzodiazepine.

11. A method for controlling administration of a therapeutic compound comprising mixing said therapeutic compound and a vanilloid receptor-1 (VR1) agonist in the same pharmaceutical composition.

12. The method of claim 11, wherein said pharmaceutical composition is suitable for ingestion.

13. The method of claim 11, wherein said pharmaceutical composition is formulated for controlled release.

14. The method of claim 11, wherein said VR1 agonist is selected from the group consisting of capsaicin, resiniferatoxin, olvanil, piperine, zingerone, anandamide, 12- and 15-(S)-hydroperoxyeicosatetraenoic acids, 5- and 15-(S)-hydroxyeicosatetraenoic acids, phorbol 12-phenylacetate 13-acetate 20-homovanillate, 2 phorbol 12,13-didecanoate 20-homovanillate, leukotriene B(4), N-(3-acyloxy-2-benzylpropyl)-N′-dihydroxytetrahydrobenzazepine, and tetrahydroisoquinoline thiourea analogs.

15. The method of claim 14, wherein said VR1 agonist is capsaicin.

16. The method of claim 14, wherein said VR1 agonist is resiniferatoxin.

17. The method of claim 11, wherein said therapeutic compound is an opioid.

18. The method of claim 17, wherein said opioid is morphine, oxycodone, or hydrocodone.

19. The method of claim 11, wherein said therapeutic compound is selected from the group consisting of morphine, oxycodone, hydrocodone, hydromorphone, levorphanol, buprenorphine, butorphanol, fentanyl, dipipanone, codeine, dihydrocodeine, tramadol, etorphine, dihydroetorphine, meperidine, methadone, propoxyphene, and heroin.

20. The method of claim 11, wherein said therapeutic compound is a cannabinoid, an amphetamine, or a benzodiazepine.

21. A method of manufacturing a pharmaceutical composition comprising mixing a therapeutic compound and a vanilloid receptor-1 (VR1) agonist.

22. The method of claim 21, wherein said pharmaceutical composition is suitable for ingestion.

23. The method of claim 21, wherein said pharmaceutical composition is formulated for controlled release.

24. The method of claim 21, wherein said VR1 agonist is selected from the group consisting of capsaicin, resiniferatoxin, olvanil, piperine, zingerone, anandamide, 12- and 15-(S)-hydroperoxyeicosatetraenoic acids, 5- and 15-(S)-hydroxyeicosatetraenoic acids, phorbol 12-phenylacetate 13-acetate 20-homovanillate, 2 phorbol 12,13-didecanoate 20-homovanillate, leukotriene B(4), N-(3-acyloxy-2-benzylpropyl)-N′-dihydroxytetrahydrobenzazepine, and tetrahydroisoquinoline thiourea analogs.

25. The method of claim 24, wherein said VR1 agonist is capsaicin.

26. The method of claim 24, wherein said VR1 agonist is resiniferatoxin.

27. The method of claim 21, wherein said therapeutic compound is an opioid.

28. The method of claim 27, wherein said opioid is morphine, oxycodone, or hydrocodone.

29. The method of claim 21, wherein said therapeutic compound is selected from the group consisting of morphine, oxycodone, hydrocodone, hydromorphone, levorphanol, buprenorphine, butorphanol, fentanyl, dipipanone, codeine, dihydrocodeine, tramadol, etorphine, dihydroetorphine, meperidine, methadone, propoxyphene, and heroin.

30. The method of claim 21, wherein said therapeutic compound is a cannabinoid, an amphetamine, or a benzodiazepine.
